(19) United States
(12) Patent Application Publication
(10) Pub. No.: US 2004/0209309 A1
(43) Pub. Date: Oct. 21, 2004

(54) Compositions and methods for the specific detection of mammalian muscle proteins

(76) Inventors: Mark Thomas Muldoon, Elkton, MD (US); Dale Vernon Onisk, Bear, DE (US); Michael Craig Brown, North East, MD (US); James W. Stave, Bear, DE (US)

Correspondence Address: JOHN S. PRATT, ESQ, KILPATRICK STOCKTON, LLP, 1100 PEACHTREE STREET, ATLANTA, GA, 30309, US

(21) Appl. No.: 10/789,433

(22) Filed: Feb. 27, 2004

Related U.S. Application Data
(60) Provisional application No. 60/450,514, filed on Feb. 27, 2003.

(51) Int. Cl. C12Q 1/68; G01N 33/53; C07K 16/18

(52) U.S. Cl. 435/7.1; 530/388.1

(57) ABSTRACT

Compositions and methods for detecting mammalian muscle proteins and for distinguishing between mammalian muscle proteins and avian muscle proteins in a sample, such as animal feed. The compositions are ligands, such as antibodies and polynucleotides; antigens for the production of ligands; and kits containing the ligands. The methods include assays that employ the ligands for the detection of mammalian muscle proteins. Preferably, the ligands bind with specificity to mammalian troponin I fast twitch or slow twitch skeletal muscle proteins.

### CROSS REFERENCE TO RELATED APPLICATIONS

This application claims the priority benefit of provisional U.S. Patent Application No. 60/450,514 filed Feb. 27, 2003.

### FIELD OF THE INVENTION

The present invention relates to the field of microbiology and more specifically relates to compositions and methods for the detection of troponin molecules.

### BACKGROUND OF THE INVENTION

Transmissible spongiform encephalopathies are caused by infection with prion pathogens. Prions have some properties in common with other infectious pathogens, but do not appear to contain nucleic acid. Prion proteins accumulate in the central nervous system where they cause neuropathologic changes and neurological dysfunction. Specific examples of transmissible spongiform encephalopathies include scrapie, which affects sheep and goats; bovine spongiform encephalopathy (BSE), which affects cattle; feline spongiform encephalopathy and chronic wasting disease of deer and elk. In humans, transmissible spongiform encephalopathies are known as kuru, Creutzfeldt-Jakob disease (CJD), Gerstmann-Straüssler-Scheinker Syndrome (GSS), fatal insomnia and variant Creutzfeldt-Jakob disease (vCJD). Variant Creutzfeldt-Jakob disease recently emerged in humans as a result of the BSE epidemic in Britain and is most probably caused by the consumption of food products derived from cattle infected with bovine spongiform encephalopathy or “mad cow disease”.

Rendered animal byproducts are commonly used as protein supplements in animal feed. Rendered animal byproducts are produced from meat byproduct materials that are undesirable for human consumption, such as, for example, bone, connective tissue, skin, hair, certain muscles, and combinations thereof. The byproducts are processed, or rendered, to facilitate their addition to the feed.

The emergence and spread of BSE in cattle, beginning in 1986, has been attributed in part to the consumption by livestock of feed containing BSE-contaminated rendered animal byproducts. Most cases have been reported in the United Kingdom. The export of contaminated bovine feed products from the United Kingdom worldwide indicates a possible global presence of BSE and hence the probability of the spread of vCJD. Consistent with these observations is the detection of BSE in most European countries, Japan and Israel. As of April 2002, 125 vCJD cases have been reported worldwide, primarily in the United Kingdom; however, cases have also been reported in Canada, France, Hong Kong, Ireland, Italy, and the United States.

In an effort to prevent, control, and eradicate BSE, several regulatory measures have been put in place worldwide. Many of these regulations prohibit or restrict the use of mammalian (i.e., bovine, porcine and equine) by-products in feed, while at the same time allowing for the use of avian by-products in feed. Accordingly, determining compliance with these legal requirements creates the need for a test to distinguish between the presence of mammalian proteins and avian proteins in feed materials, and more specifically, between mammalian muscle proteins and avian muscle proteins.

While binding assays do exist that detect certain proteins found in muscle tissue, such as titin, troponin, and muscle extracts (see, for example, Ansfield, M. (1994), Production of a Sensitive Immunoassay for Detection of Ruminant Proteins in Rendered Animal Material Heated to >130° C., Food Agric. Immunol. 6, 419-433; Chen, F-C; Hsieh, P.; Bridgeman, R. C. (2002), Monoclonal Antibodies Against Troponin I for the Detection of Rendered Muscle Tissues in Animal Feedstuffs, Meat Sci. 62, 405-412; Pospiech, E. Greaser, M. L.; Mikolajczak, Chiang, W.; Krzywdzinska, M. (2002), Thermal Properties of Titin from Porcine and Bovine Muscles, Meat Sci. 62, 187-192; and AGRI-SCREEN Ruminant in Feed Strip Test available from Neogen Corporation, (Lansing, Mich.)), these assays suffer from the inability to specifically recognize mammalian proteins, such as bovine and porcine muscle proteins. The non-specificity of presently available assays causes them to detect proteins from a variety of species including avian muscle proteins, thereby producing positive assay results in samples that are devoid of mammalian proteins.

Accordingly, what is needed is an assay that overcomes the problems of prior art assays that fail to distinguish between mammalian and avian molecules in animal feed, thereby maximizing detection to promote public health efforts to prevent the spread of prion disease.

### SUMMARY OF THE INVENTION

The present invention overcomes the problems of the prior art by providing compositions and methods for detecting mammalian muscle proteins in a sample such as animal feed. The compositions and methods are assay reagents, immunogens, and assays specific for mammalian proteins. The assay reagents are specific for mammalian muscle proteins and are reactive with and bind to mammalian proteins but lack binding reactivity with avian muscle proteins. The preferred assay reagents are antibodies. The immunogens are peptides useful for producing the antibodies. Particularly preferred peptides are set forth herein. The assays are highly specific and are therefore able to distinguish mammalian from non-mammalian muscle proteins in a sample. The preferred muscle protein to be detected is troponin.

The ligands are collectively assembled in a kit with conventional assay reagents for the detection of a mammalian protein, such as troponin molecules, in a sample. A preferred kit contains either monoclonal antibodies, polyclonal antibodies, or both, and optionally includes a standard for determining the presence or relative concentration of mammalian muscle protein in the sample.

It is therefore an object of the present invention to provide assay reagents or ligands (such as antibodies and polynucleotides), antigens or immunogens, assay methods, and kits for the detection of mammalian troponin molecules in a sample, particularly an agricultural sample, such as animal feed.

It is a further object of the present invention to provide antibody ligands specific for mammalian troponin molecules and not immunoreactive with avian troponin I molecules.

It is another object of the present invention to provide antibody ligands specific for bovine and porcine troponin molecules and not immunoreactive with avian troponin I molecules.

It is still another object of the present invention to provide antibody ligands specific for fast twitch skeletal muscle troponin I proteins and/or slow twitch skeletal muscle troponin I proteins.

These and other objects of the present invention will become apparent after reading the following detailed description of the disclosed embodiments and the appended claims.

### BRIEF DESCRIPTION OF THE DRAWING

FIG. 1 shows an amino acid sequence comparison between human cardiac troponin I protein, fast twitch skeletal muscle troponin protein from rabbit, rat, mouse, human, goat, chicken and quail and slow twitch skeletal muscle troponin protein from mouse, rat, human, rabbit, goat and quail. The single underlined amino acids are those that differ between the avian troponin sequences and consensus mammalian troponin sequences. The double underlined amino acids are amino acids that differ in mammals but are the same in avian troponin sequences.

### DETAILED DESCRIPTION OF THE PREFERRED EMBODIMENTS

Ligands, antigens, assay methods, and kits for the detection of mammalian muscle protein in a sample are provided. The ligands are specific for mammalian muscle proteins, preferably mammalian troponin molecules, and more preferable mammalian troponin I molecules. The ligands are non-reactive with the corresponding muscle proteins of non-mammals, such as avian species. The antigens are mammalian muscle peptides useful for producing the ligands described herein. The assays distinguish between samples containing mammalian muscle protein, samples containing avian muscle protein, and samples containing both mammalian and avian muscle protein. Kits for performing such assays are provided. In a preferred embodiment, the sample is an agricultural sample such as animal feed. In a further preferred embodiment, the animal feed is suspected of containing animal by-products.

As used herein, the term “animal feed” refers to any substance provided to an animal for nourishment. The term “animal by-product” as used herein means parts or portions of animals, including, but not limited to portions discarded from the preparation of meat products from animals for human consumption. This term includes, for example, bone, connective tissue, skin, hair, certain muscles, and combinations thereof.

Troponin proteins are found in animal muscle tissue and are used herein as identifiers of animal by-products in animal feed and other samples. Three types of troponin proteins form a complex that is involved in the regulation of muscle contraction at the thin filament level. Troponin I, troponin C, and troponin T are the three types of troponin proteins present in the complex. Troponin I proteins can be subdivided into several isoforms that include cardiac troponin I, slow twitch skeletal muscle troponin I, and fast twitch skeletal muscle troponin I.

In a preferred embodiment, the ligand provided herein is specific for mammalian troponin I. In a more preferred embodiment, the ligand provided herein is specific for a slow twitch skeletal muscle troponin I molecule (hereinafter referred to as “ST troponin I”) or a fast twitch skeletal muscle troponin I molecule (hereinafter referred to as “FT troponin I”). In a still more preferred embodiment, the ligand is specific for a mammalian ST troponin I molecule and/or a mammalian FT troponin I molecule, and is not specific for an avian troponin molecule. The term “non-specific” as used herein means that the ligand is non-reactive, fails to bind, or binds minimally and fails to produce a positive, detectable signal, to an avian troponin molecule, particularly an avian troponin I molecule. In a still more preferred embodiment, the ligand is specific for a particular region within a FT or ST troponin I molecule, which region is conserved between several mammalian troponin I molecules, but not conserved between mammalian and avian troponin I molecules. In a still more preferred embodiment, the ligand is specific for bovine and porcine FT and/or ST troponin I molecules and is non-reactive with avian troponin I molecules.

Antigens

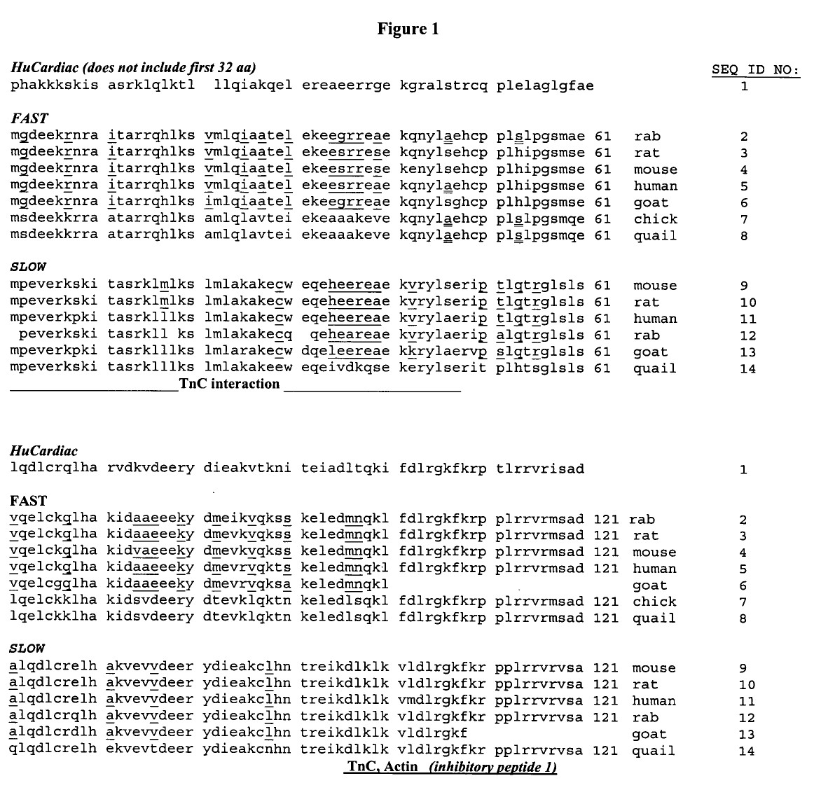

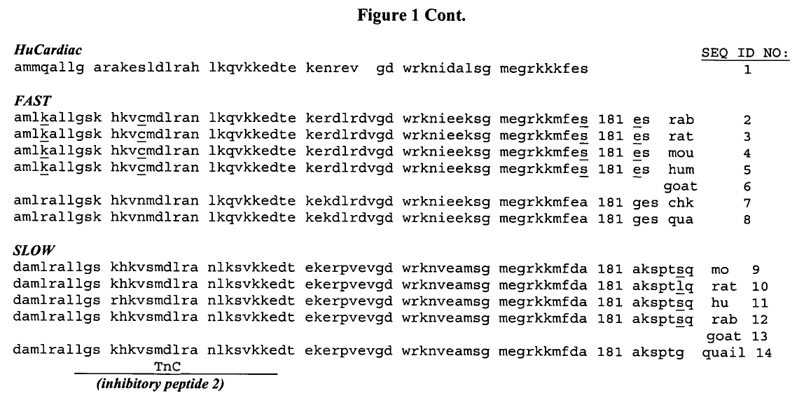

Peptides and amino acid sequences of peptides are provided herein for use in the production of ligands specific for mammalian muscle protein. Several exemplary amino acid sequences of peptides from cardiac, fast twitch, and slow twitch troponin I molecules, including those from rabbit, rat, mouse, human, goat, chicken and quail are shown in FIG. 1 and SEQ ID NOS:1-14. In FIG. 1, amino acids that differ between avian sequences and a consensus mammalian species are underlined once. Peptides having the amino acid sequences containing these differences or peptides having amino acid sequences substantially homologous to these peptides are useful for producing ligands specific for mammalian troponin I. In FIG. 1, areas where some mammals differ from other mammals, but are the same as avian are underlined twice. Peptides having the amino acid sequences containing these regions of differences are less desirable for the production of ligands specific for mammalian troponin I. It will be understood by those skilled in the art that these examples are not limiting.

As used herein, the terms “mammalian” and “mammal” include, but are not limited to, bovine, ovine, porcine, equine, murine and primate animals. The term “mammal” particularly includes ruminant animals such as cows. The term “avian” refers to an animal in the Aves class that is characterized as a warm-blooded, egg-laying vertebrate primarily adapted for flying. Avians include, without limitation, Ratites, Psittaciformes, Falconiformes, Piciformes, Strigiformes, Passeriformes, Coraciformes, Ralliformes, Cuculiformes, Columbiformes, Galliformes, Anseriformes, and Herodiones. Accordingly, in one embodiment, the ligand is specific for a troponin I molecule derived from a bovine, ovine, porcine, equine, murine and primate animal, or any combination thereof, and is non-reactive with a troponin I molecule derived from an avian animal such as, but not limited to, a poultry animal such as a chicken, duck, goose, turkey, pigeon, quail, or the like.

Specific peptides for use in the production of ligands specific for mammalian troponin and non-reactive, or minimally reactive, with non-mammalian troponin are described in more detail below.

Ligands

The term “ligand”, as used herein, refers to a molecule that binds to an epitope or binding site and includes antibodies, proteins, peptides, polypeptides, amino acids, polynucleotides, carbohydrates, sugars, lipids, organic molecules, polymers and the like. In one embodiment, the ligand is an antibody, and the troponin I molecule is a troponin I polypeptide. Both polyclonal and monoclonal antibodies are useful as the ligands provided herein. In another embodiment, the ligand is a polynucleotide sequence and the troponin I molecule is a troponin I polynucleotide sequence that hybridizes with the ligand polynucleotide, preferably under stringent hybridization conditions. Preferably, the troponin I polynucleotide sequence encodes one of the mammalian FT or ST troponin I peptides or polypeptides shown in FIG. 1 and designated as SEQ ID NOS:2-6,9-13 and 15-35.

The antibody ligands of the present invention may be produced by the administration of one or more troponin proteins or one or more of the troponin I peptides or polypeptides described herein to an animal under conditions effective to induce an antigenic response and subsequent isolation of the antibodies from a biological fluid of the animal. Alternatively, the ligands are polynucleic acid molecules that hybridize to nucleotide sequences encoding the amino acid sequences described herein and are produced using isolation, recombinant or synthetic methods well known to one of ordinary skill in the art.

In a preferred embodiment, the ligands described herein are monoclonal or polyclonal antibodies produced by immunizing an animal with an immunogenic composition comprising a troponin I molecule. Preferred troponin I molecules are FT and ST troponin I molecules. In some embodiments, the antibody ligands are produced by immunizing an animal with an immunogenic composition comprising one or more FT or ST troponin I polypeptides shown in SEQ ID NOs:2-35 and described in more detail below.

The terms “protein”, “peptide”, “polypeptide” and “oligopeptide” are used interchangeably and refer to chains of amino acids (typically L-amino acids) whose alpha carbons are linked through peptide bonds. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the protein, or to the alpha-amino group (imino group when participating in a peptide bond) of an amino acid at any other location within the protein. Similarly, the term “carboxy terminus” (abbreviated C-terminus) refers to the free carboxyl group on the amino acid at the carboxy terminus of a protein, or to the carboxyl group of an amino acid at any other location within the protein.

Preferred mammalian polypeptides used to generate the antibody ligands described herein are provided in SEQ ID NOS:2-6 and 9-13, wherein SEQ ID NOS:2-6 correspond to amino acid sequences of various mammalian FT troponin I proteins and SEQ ID NOS:9-13 correspond to various mammalian ST troponin I proteins. In a further preferred embodiment, the ligand is specific for a particular region within a FT or ST troponin I protein, which region is conserved between several mammalian troponin I proteins, but not conserved between mammalian and avian troponin I proteins. Immunization of an animal with one or more of these mammalian polypeptides results in the production of antibody ligands that are specific for mammalian FT and/or ST troponin I proteins and are not specific for avian troponin proteins. In a preferred embodiment, the antibody ligands are specific for porcine and bovine FT and/or ST troponin I proteins and do not cross react with avian troponin proteins.

Examples of polypeptide regions within FT troponin proteins that are conserved between several mammalian troponin I molecules, but not conserved between mammalian and avian troponin I molecules are shown in FIG. 1. Specific amino acids contained in this region are underlined once. These polypeptide regions are useful for generating antibody ligands that are specific for mammalian FT troponin proteins and not specific for, or non-cross reactive with, avian troponin I proteins. More particularly, amino acids 21 through 59 in FIG. 1, or VMLQIAATELEKEESRRESEKENYLSEHCPPLHIPGSMS (SEQ ID NO:15) and IMLQIAATELEKEEGRREAEKQNYLSGHCPPLHLPGSMS (SEQ ID NO:16), are useful for generating antibodies specific for mammalian troponin. Within this area, amino acids 21 through 40 in FIG. 1, or VMLQIAATELEKEESRRESE (SEQ ID NO: 17) and IMLQLAATELEKEEGRREAE (SEQ ID NO:18), are particularly useful as is the sequence from amino acids 45 through 59 in FIG. 1, or SEHCPPLHIPGSMS (SEQ ID NO:19) and SGHCPPLHIPGSMS (SEQ ID NO:20). Within the sequence from amino acids 21 through 40 in FIG. 1, the sequence from amino acids 33 through 40, or EKEESRRESE (SEQ ID NO:21) and EKEEGRREAE (SEQ ID NO:22), are again particularly useful for generating antibodies specific for mammalian troponin I proteins that are not specific for avian troponin I proteins. Additionally, for FT troponin I proteins, amino acids 61 through 97 in FIG. 1, or VQELCKQLHAKIDAAEEEKYDMEVKVQKSSKELEDMN (SEQ ID NO:23) and VQELCGQLHAKIDAAEEEKYDMEVRVQKSAKELEDMN (SEQ ID NO:24), are useful for generating antibodies specific for mammalian troponin I proteins. Particularly useful are amino acids 61 through 82 in FIG. 1, or VQELCKQLHAKIDAAEEEKYDM (SEQ ID NO:25) and VQELCGQLHAKIDAAEEEKYDM (SEQ ID NO:26), and more useful, amino acids 67 through 77, or KQLHAKIDAAEE (SEQ ID NO:27) and GQLHAKIDAAEE (SEQ ID NO:28). Accordingly, preferred antibody ligands that are specific for one or more of the mammalian FT troponin I polypeptides are specific for one or more polypeptide sequences provided in SEQ ID NOS:15-28.

In addition to the mammalian FT troponin I polypeptide regions, FIG. 1 also shows ST troponin I polypeptide regions that are conserved between several mammalian troponin I molecules, but not conserved between mammalian and avian troponin I molecules. Polypeptides in these regions are useful for generating antibody ligands that are specific for mammalian ST troponin I proteins and not reactive with avian troponin I proteins. More particularly, within ST troponin I proteins, amino acids 28 through 76 in FIG. 1, or ECWEQEHEEREAEKVRYLAERIPTLQTRGLSLSALQDLCRELHAKVEVV (SEQ ID NO:29) and ECWEQELEEREAEKKRYLAERIPTLQTRGLSLSALQDLC RDLHAKVEVV (SEQ ID NO:30), are useful for generating antibodies specific for mammalian ST troponin I proteins. Within this area in particular, sequences from amino acids 70 through 76 HAKVEVV (SEQ ID NO:31) are useful as is the sequence from amino acids 28 through 44, or ECWEQEHEEREAEKVRY (SEQ ID NO:32) and ECWEQELEEREAEKKRY (SEQ ID NO:33), and amino acids 49 through 55, or IPTLQTR (SEQ ID NO:34) and IPSLQTR (SEQ ID NO:35). Accordingly, antibody ligands that are specific for one or more of the ST troponin I polypeptides are specific for one or more polypeptide sequences provided in SEQ ID NOS:29-35.

In certain embodiments, the present invention also includes ligands that are specific for avian troponin I molecules. Non-limiting examples of avian troponin I proteins are shown in FIG. 1 and designated as SEQ ID NOS:7, 8 and 14.

In other embodiments, the ligand is an antibody produced by immunizing an animal with a molecule or substance that differs structurally from a polypeptide provided in SEQ ID NOS:2-6, 9-13 and 15-35, but which molecule or substance is recognized by an antibody that is specific for a polypeptide provided in SEQ ID NOS: 2-6, 9-13 and 15-35. Examples of such molecules and substances include, but are not limited to: fragments of the polypeptides provided in SEQ ID NOS: 2-6, 9-13 and 15-35; synthetic molecules that differ structurally from the polypeptides provided in SEQ ID NOS: 2-6, 9-13 and 15-35, but that contain one or more epitopes from one or more of these polypeptides; molecules that contain a deletion from, addition to, or substitution of a portion of a polypeptide provided in SEQ ID NOS: 2-6, 9-13 and 15-35 where such deletion, addition, or substitution does not impair the ability of the epitope to be recognized by an antibody that also recognizes a polypeptide provided in SEQ ID NOS: 2-6, 9-13 and 15-35; and constructs such multiple antigenic peptides comprising multiple epitopes or constructs in which a portion of polypeptide provided in SEQ ID NOS: 2-6, 9-13 and 15-35 is fused to an immunogenic carrier molecule. In the foregoing embodiments, the compositions used to immunize the animal optionally contain one or more buffers or adjuvants or both.

Polyclonal antibodies are advantageous in that they can be produced at low cost and, in some cases, superior sensitivity, over monoclonal antibodies. Therefore, under certain circumstances, the preferred antibody provided herein is a polyclonal antibody. It will be understood by those skilled in the art that polyclonal antibodies immunoreactive with mammalian troponin protein are generated by immunizing an animal with the whole troponin complex, troponin I protein, troponin T protein, troponin C protein or fragments thereof. Use of the troponin complex or a whole troponin protein to produce polyclonal antibodies will result in the production of a mixture of both mammalian specific and non-specific (cross reactive with avian) antibodies, whereas immunization with one or more of the specific peptides set forth above will result in the production of polyclonal antibodies that are specific for mammalian troponin as described above. However, if the whole troponin complex or proteins are used, the resulting polyclonal mixture can be processed to separate the mammalian specific antibodies from the other antibodies in the mixture by using the antigens described herein. For example, the peptide antigens provided herein are coupled to an affinity matrix, or column, and the polyclonal antibody mixture is purified by passing it through the column in accordance with protein separation methods well known to those skilled in the art. Mammalian-specific polyclonal antibodies in the mixture will bind to the peptides and be retained on the column while the non-specific polyclonal antibodies pass through the column and can be collected or discarded. The bound antibodies can then be eluted from the column using a predetermined eluant.

Although the antibodies isolated directly from serum as described above are polyclonal antibodies, the antibodies described herein also include monoclonal antibodies. The term “antibodies” as used herein includes monoclonal, polyclonal, chimeric, single chain, bispecific, simianized, and humanized antibodies as well as Fab fragments, including the products of a Fab immunoglobulin expression library. An antibody is “specific for” a particular protein when the antibody binds to the protein with sufficient affinity and avidity to result in the production of a detectable antibody-antigen complex.

Various methods can be used to generate monoclonal antibodies. Several methods for generating monoclonal antibodies are well known to those skilled in the art. One method is a modified version of the method of Kearney, et al., J. Immunol. 123:1548-1558 (1979), which is incorporated by reference herein. Briefly, animals such as mice or rabbits are inoculated with the immunogen in adjuvant, and spleen cells are harvested and mixed with a myeloma cell line, such as P3X63Ag8,653. The cells are induced to fuse by the addition of polyethylene glycol. Hybridomas are chemically selected by plating the cells in a selection medium containing hypoxanthine, aminopterin and thymidine (HAT). Hybridomas are subsequently screened for the ability to produce anti-troponin I monoclonal antibodies. Hybridomas producing antibodies are cloned, expanded and stored frozen for future production.

In some embodiments, antibodies are generated by immunizing an animal with an immunogenic amount of a troponin I antigen emulsified in an adjuvant such as Freund's complete adjuvant, administered over a period of weeks in intervals ranging between two weeks and six weeks. In a preferred embodiment, the first immunization includes Freund's complete adjuvant and subsequent immunizations including Freund's incomplete adjuvant are made at biweekly to monthly intervals thereafter. Test bleeds are preferably taken at fourteen-day intervals between the second and third immunizations and production bleeds at monthly intervals thereafter. Antibodies may be isolated from the serum, or spleen cells from the immunized animal may be fused with myeloma cells to make hybridomas that express the antibodies in culture.

In some embodiments, hybridomas are produced by fusing a culture of immune lymphocytes with modified myeloma cells, using polyethylene glycol (PEG) with modified myeloma cells in a defined tissue culture medium, such as HAT (hypoxanthine, aminopterin, thymidine), is capable of providing a variety of fusion products, such as s-s, s-m, and m-m (with s=splenocyte and m=myeloma cell). Within the tissue culture medium, the s-s fusion product normally has a short lifetime and dies within days. Also, the m-m fusion product has a very short lifetime in the tissue culture medium used since the metabolic components needed for DNA synthesis are lacking. However the s-m fusion product (or hybridoma) survives in tissue culture and retains the antibody-producing characteristics of the splenocyte parent, and the high rate of growth and relative immortality of the myeloma cell parent. These hybridoma cell lines replicate readily in culture producing daughter cells that provide a reproducible, homogeneous, and consistent supply of the monoclonal antibody of the present invention. Selection of the appropriate cell line provides the monoclonal antibody of a preferred embodiment of the present invention.

Ligand Labeling

In a preferred embodiment, the ligand described herein is labeled to allow detection of a troponin I molecule in a sample. For example, in some embodiments the labeled ligand is combined with the sample, and the labeled ligand-troponin I complex is detected. The ligand is, for example, labeled during ligand production, such as during peptide synthesis, or a label is conjugated to the ligand by joining it to the ligand, either covalently or non-covalently. Alternatively, a binding molecule specific for the ligand, such as an antibody, is labeled and the complex is detected indirectly.

A wide variety of labels and conjugation techniques are known and are reported extensively in both the scientific and patent literature. Any label and any conjugation technique may be used. Suitable labels include radioactive molecules, enzymes, substrates, cofactors, inhibitors, fluorescent moieties, chemiluminescent moieties, magnetic particles, and the like. The particular label or detectable group used in the assay is not a critical aspect of the invention. The detectable group can be any material having a detectable physical or chemical property. Such detectable labels have been well developed and, in general, any label useful in such methods can be applied to the present method. Thus, a label is any composition detectable by spectroscopic, photochemical, biochemical, immunochemical, electrical, optical or chemical means. Useful labels in the present invention include fluorescent dyes (e.g., fluorescein isothiocyanate, Texas red, rhodamine, and the like), radiolabels (e.g., ³H, ¹²⁵I, ³⁵S, ¹⁴C or ³²P), enzymes (e.g., LacZ, CAT, horseradish peroxidase, alkaline phosphatase and others, commonly used as detectable enzymes, either in an EIA or in an ELISA), and colorimetric labels such as colloidal gold or colored glass or plastic (e.g., polystyrene, polypropylene, latex, etc.) beads. The label may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions.

Non-radioactive labels are often attached by indirect means using binding partner molecules. In one embodiment, a first binding partner molecule (e.g., biotin) is covalently bound to the ligand described above. The biotin molecule then binds to a second binding partner molecule (e.g., streptavidin), which is either inherently detectable or covalently bound to a signal system such as a detectable enzyme, a fluorescent compound, or a chemiluminescent compound. Any two binding partner molecules that will function together can be used. In some embodiments in which the first binding partner has a natural second binding partner, for example, biotin, thyroxine, and cortisol, it is used in conjunction with the labeled, naturally occurring second binding partner. Alternatively, any haptenic or antigenic compound can be used in combination with an antibody ligand.

In some embodiments, the ligands are conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol.

Any of the labels discussed above for ligands in general may be used for antibody ligands. Some preferred labels for use in immunoassays that include antibody ligands are generally known to those skilled in the art and include enzymes, radioisotopes, and fluorescent, luminescent and chromogenic substances including colored particles such as colloidal gold and latex beads. Furthermore, in some embodiments, the antibody is labeled indirectly by reaction with labeled substances that have an affinity for immunoglobulin, such as protein A or G or second antibodies. In some embodiments, the antibody is conjugated with a second substance and detected with a labeled third substance having an affinity for the second substance conjugated to the antibody. For example, in one embodiment the antibody is conjugated to biotin and the antibody-biotin conjugate is detected using labeled avidin or streptavidin. In one embodiment, the antibody is conjugated to a hapten and the antibody-hapten conjugate is detected using labeled anti-hapten antibody. These and other methods of labeling antibodies and assay conjugates are well known to those skilled in the art.

In some embodiments, the antibody ligand is immobilized on a solid phase to facilitate detection of the troponin protein. Any solid phase that will allow immobilization may be used. It will be understood by those skilled in the art that examples of solid phases include nitrocellulose, latex, polystyrene, polyethylene, polypropylene, polycarbonate or any solid plastic material in the shape of test tubes, beads, microparticles, dip-sticks or the like. A solid phase also includes glass beads, glass test tubes and any other appropriate shape made of glass. Preferably, the solid phase is a nitrocellulose strip.

Assays

A highly sensitive assay employing a ligand specific for a mammalian troponin molecule and lacking cross-reactivity for an avian troponin molecule, as described above, is provided. The assay is useful for detecting the presence or amount of a mammalian troponin molecule in a sample, and therefore is useful for detecting the presence of a mammalian animal by-product in a sample. A preferred sample is animal feed. The assay is particularly useful for assessing and maintaining compliance with governmental regulations and guidelines regarding the acceptable animal by-products to be contained in animal feed in an attempt to reduce or eliminate the spread of infectious prion disease.

A preferred assay is an immunoassay that employs an antibody specific for a mammalian FT and/or ST troponin I molecule and not cross-reactive with avian troponin I molecule. In a more preferred embodiment, the antibody is specific for one or more polypeptides provided in SEQ ID NOS:2-6, 9-13 and 15-35. In some embodiments, the antibody is specific for bovine and porcine troponin I proteins and lacks immunoreactivity with avian troponin proteins. In some embodiments of the present invention, the assay also employs an antibody specific for an avian troponin I protein or polypeptide. Non-limiting examples of avian troponin I polypeptides are provided in SEQ ID NOS:7, 8 and 14. The avian-specific antibody is useful as a negative control.

In embodiments involving immunoassays, the antibody and assay conjugates may be employed in any heterogeneous or homogeneous, sandwich or competitive immunoassay for the detection of a troponin molecule in a sample. The antibody is labeled (directly or indirectly) with a detectable label, coupled to a solid phase, or both. A preferred solid phase is nitrocellulose, and more preferably, a nitrocellulose strip. Any method of labeling or coupling may be used. Methods for coupling antibodies to solid phases are well known to those skilled in the art. In accordance with the immunoassay method, the sample suspected of containing mammalian by-products, which should also include mammalian troponin I proteins, is reacted with the antibody for a sufficient amount of time under conditions that promote the binding of antibody to a mammalian troponin protein in the sample. It will be understood by those skilled in the art that the immunoassay reagents and sample may be reacted in different combinations and orders. A physical means is employed in some embodiments to separate reagents bound to the solid phase from unbound reagents. Examples of such means include, but are not limited to filtration of particles, decantation of reaction solutions from coated tubes or wells, magnetic separation, capillary action, and other means known to those skilled in the art. It will also be understood that a separate washing of the solid phase may be included in the method.

After reaction, the existence, concentration, or both, of the troponin protein is determined by the signal generated by the label. The presence or location of the signal may be an indicator for the presence of the troponin protein in the sample. In one embodiment involving a sandwich immunoassay, a ligand such as an antibody is fixed to a substrate (or solid phase) and the sample is contacted with the substrate under conditions effective to cause the antibody to bind a troponin protein in the sample. The substrate having antibody fixed thereupon is also contacted (either subsequently or simultaneously with its contact with the sample) with free or unfixed antibody or ligand that is labeled under conditions effective to cause the labeled antibody or ligand to bind the troponin protein that has already bound to the fixed antibody. The substrate is then washed to remove any unbound antibodies or ligand and the presence and/or concentration of the troponin protein is indicated by the presence and/or strength of the label signal. In another embodiment involving a direct assay, the sample is placed under conditions effective to cause any troponin molecules in the sample to become fixed on a substrate. The substrate is then contacted with labeled ligand such as an antibody under conditions effective to cause binding of the antibody to any bound troponin protein. The substrate is then washed to remove any unbound antibodies and the presence and/or concentration of the troponin protein is indicated by the presence and/or strength of the label signal. The foregoing are simply examples of assays and any immunoassay method may be used, including other types of direct and indirect assays as well as competitive assays.

Detection of labels may occur by any method. Examples of known methods include, but are not limited to immunoblotting, western analysis, gel-mobility shift assays, fluorescent in situ hybridization analysis (FISH), tracking of radioactive or bioluminescent markers, nuclear magnetic resonance, electron paramagnetic resonance, stopped-flow spectroscopy, column chromatography, capillary electrophoresis, or other methods which track a molecule based upon an alteration in size and/or charge. Additionally, any means may be used to detect labels. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels are detected by providing appropriate substrates for the enzyme and detecting the resulting reaction product. Simple colorimetric labels may be detected simply by observing the color associated with the label. Thus, in various dipstick assays, conjugated gold often appears pink, while various conjugated beads appear the color of the bead.

In some embodiments, the ligands described herein are used to detect troponin molecules extracted into solution from a solid material. For example, a solid sample can be extracted with an aqueous liquid, an organic solvent or a critical fluid and the resultant supernatant can be contacted with the ligand. Examples of solid samples include animal-derived products, particularly those that have been exposed to rendered animal byproducts (e.g., feed).

Preferred detection methods include a direct or indirect enzyme-linked immunosorbent assay (ELISA) using a secondary antibody such as a peroxidase-conjugated goat anti-mouse antibody or a direct or indirect immunofluorescence assay using a secondary antibody such as a fluorescein isothiocyanate (FITC)-conjugated goat anti-mouse antibody.

In some embodiments, the concentration of a troponin molecule in the sample is determined either by comparing the intensity of the color produced by the sample to a color card or by using a reflectometer.

In some embodiments the resulting reaction mixture, or combination of antibody and sample, is prepared in a solution that optimizes antibody-troponin binding kinetics. An appropriate solution is an aqueous solution or buffer. The solution is preferably provided under conditions that will promote specific binding, minimize nonspecific binding, solubilize troponin, stabilize and preserve reagent reactivity, and may contain buffers, detergents, solvents, salts, chelators, proteins, polymers, carbohydrates, sugars, and other substances known to those skilled in the art.

The reaction mixture solution is reacted for a sufficient amount of time to allow the antibody to react and bind to the troponin protein to form an antibody-troponin complex. The shortest amount of reaction time that results in binding is desired to minimize the time required to complete the assay. An appropriate reaction time period for an immunochromatographic strip test is less than or equal to 20 minutes or between approximately one minute and 20 minutes. A reaction time of less than five minutes is preferred. Most preferably, the reaction time is less than three minutes. By optimizing the reagents, binding may be substantially completed as the reagents are combined.

The reaction is performed at any temperature at which the reagents do not degrade or become inactivated. A temperature between approximately 4° C. and 37° C. is preferred. The most preferred reaction temperature is ambient or room temperature (approximately 25° C.). Strip tests are comprised of multiple porous components, membranes and filters through which liquid sample is drawn by capillary action. A preferred strip is a nitrocellulose strip. Troponin molecules in the sample react with the test reagents contained within the test strip as they traverse the length of the strip. In one embodiment in which the goal is to detect troponin I in feed, the feed is ground into a powder and the protein extracted from the powder with a liquid that is then separated from the solid material and assayed using the test. The liquid is applied to the chromatographic strip, and the troponin molecule migrates toward the distal end of the strip. As it migrates down the strip, the troponin molecule reacts with reagents applied to or immobilized on the strip causing a detectable signal product. Detection of the signal indicates the presence of mammalian troponin in the sample.

Assay Kit

An assay kit for the detection of a troponin molecule in a sample contains one or more of the ligands described above. Preferably the assay kit is an immunoassay kit containing an antibody specific for one or more mammalian troponin molecules provided herein, more preferably bovine and porcine troponin I proteins, and the antibody lacks immunoreactivity with avian troponin I proteins. In a preferred embodiment, the antibody is specific for one or more epitopes or polypeptides as provided in SEQ ID NOS:2-6, 9-13 and 15-35. In some embodiments, the kit also contains an antibody specific for an avian troponin I protein, which is useful as a negative control. Non-limiting examples of avian troponin I proteins are provided in SEQ ID NOS:7, 8 and 14.

The kit may additionally contain equipment for obtaining the sample, a vessel for containing the reagents, a timing means, a buffer for diluting the sample, and a colorimeter, reflectometer, or standard against which a color change may be measured. In one embodiment, the antibody is collectively assembled in a kit with conventional immunoassay reagents for detection of a troponin molecule. The kit may optionally contain both monoclonal and polyclonal antibodies and a standard for the determination of the presence of a troponin molecule in a sample. The kit containing these reagents provides for simple, rapid, on site detection of mammalian troponin molecules in a sample, and thereby provides for simple, rapid, on site detection of mammalian by-products in a sample.

In a preferred embodiment, the reagents, including the antibody are dry. Addition of aqueous sample to the strip results in solubilization of the dry reagent, causing it to react.

The invention will be described in greater detail by way of specific examples. The following examples are offered for illustrative purposes, and are intended neither to limit nor define the invention in any manner.

### EXAMPLE 1

Detection of Mammalian Troponin I in a Feed Sample using a Mammalian Troponin I (MT1) Antibody

Feed samples known to contain mammalian by-products are obtained and protein is extracted from the sample using methods well-known to those of skill in the art. Mammalian Troponin I is detected in the extract using MT1 and MT2 antibodies in sandwich ELISA procedures, direct ELISA procedures and/or a lateral flow immunochromatography assay as described below. The MT1 and MT2 antibodies are specific for mammalian troponin I proteins, and more specifically, mammalian ST and FT troponin I proteins and are not immunoreactive with avian troponin I proteins. The MT1 antibody is able to recognize several different types of mammalian troponin I proteins including those from cow and pig.

Sandwich ELISA Procedures.

Extracts are prepared using the procedures above. Extracts are then diluted with PBS-T containing 0.1% BSA (assay buffer) to prepare dilutions contain extract in amounts of 0.00001%, 0.00010%, 0.00100%, 0.01000%, 0.10000%, 1.00000%, and 10.00000% (each percentage v/v) for each extract. MT1 antibody is coated on microtiter plates at 2.5 μg/mL in PBS. The plates are incubated overnight at 4° C. then washed with PBS-T. The plates are blocked with 100 μL Stabilcoat (Surmodics, Inc. Eden Prarie, Minn.) overnight at 4° C. The plates are washed with PBS-T. Diluted extracts of each concentration (100 μL) are incubated on plates for 1 hour at room temperature and the plate is then washed. One hundred microliters per well of MT2 antibody conjugated to horseradish peroxidase diluted in assay buffer is added and the plate was incubated for one hour at room temperature. The plate is then washed. One hundred microliters per well of TMB (available from Moss Inc., Pasadena, Md.) is added to the plates and color development is measured at 650 nm using a microtiter plate reader.

Direct Bind ELISA Procedures.

The diluted feed stock extract containing mammalian troponin protein is subjected to direct bind ELISA using the following procedures. Microtiter plates are coated with 100 μL/well of the diluted feed stock extracts. The plates are incubated overnight at 4° C. and then washed with PBS containing 0.05% (v/v) Tween 20 (PBS-T). Plates are blocked with 120 μL PBS-T containing 1% casein (PCT) for one hour at room temperature and washed with PBS-T. One hundred microliters per well of hybridoma MT1 MAb purified (using the Protein A method) or supernatant are added and the plates incubated for one hour at room temperature and then washed. Rabbit anti-mouse IgG conjugated to horseradish peroxidase diluted in PCT is added to the wells and the plate is incubated for one hour at room temperature. The plates are washed. One hundred microliters per well of TMB (Moss, Inc. Pasadena, Md.) are added to the plates and color development is measured at 650 nm using a microtiter plate reader.

Preparation of a Lateral Flow Immunochromatography Assay

Monoclonal MT1 antibody (test line) is sprayed at 1 mg/mL in PBS onto a nitrocellulose membrane (Millipore, Bedford, Mass. Cat No. HF07054500) using a Biodot XYZ3000-dispensing platform sprayer (Irvine, Calif.). Goat anti-mouse IgG (Lampire Biological Labs, Pipersville, Pa.) is sprayed as the control line at 1 mg/mL in PBS. Monoclonal MT2 antibody is conjugated to colloidal gold (BBI, Cardiff, UK; 40 nm) using standard methods (Beesley, J. E. (1989). Colloidal gold: A new perspective for cytochemical marking. Oxford University Press, New York) and dried onto polyester pads (Reemay 2033, Ahlstrom, Mt. Holly Springs, Pa.). Sprayed nitrocellulose membrane and MAb-gold-treated polyester pads are laminated onto plastic backing. A sample filter paper is placed below the gold pad at the sample application end of the strip. A wicking paper is placed above the membrane to facilitate continuous capillary flow. The assembly is cut into test strips using a guillotine cutter.

For sample analysis, 500 μL of liquid sample is placed into a 1.8 mL microcentrifuge tube. The test strip is placed into the vial where only the sample filter pad contacts the sample. The test strip is allowed to develop in the sample for ten minutes. Following ten minutes, the test strip is removed from the sample and the results are interpreted. If two lines are present, the result is positive. If one line is present (at the control zone), the result is negative.

Although methods and materials similar or equivalent to those described herein can be used in the practice or testing of the present invention, suitable methods and material are described above. All publications, patent applications, patents and other cited references mentioned herein are incorporated by reference in their entirety. In addition, the materials, methods, and examples are illustrative only and not intended to be limiting.

The foregoing description is provided for describing various embodiments relating to the invention. Various modifications, additions and deletions may be made to these embodiments and/or structures without departing from the scope and spirit of the invention.

What is claimed is:

1. A ligand specific for mammalian troponin, wherein the ligand comprises a molecule that binds to a mammalian troponin molecule, but not an avian troponin molecule.

2. The ligand of claim 1, wherein the mammalian troponin molecule is a troponin I molecule.

3. The ligand of claim 1, wherein the mammalian troponin molecule is selected from the group consisting of a slow twitch skeletal muscle troponin I molecule and a fast twitch skeletal muscle troponin I molecule.

4. The ligand of claim 1, wherein the ligand is an antibody and the troponin molecule is a polypeptide.

5. The ligand of claim 1, wherein the ligand is an antibody produced by immunizing an animal with a peptide having an amino acid sequence selected from the group consisting of SEQ ID NOS:2-6, 9-13, and 15-35.

6. The ligand of claim 1, wherein the ligand binds to a peptide having an amino acid sequence selected from the group consisting of SEQ ID NOS:2-6, 9-13, and 15-35.

7. The ligand of claim 1, wherein the ligand binds to a nucleic acid molecule encoding a peptide having an amino acid sequence selected from the group consisting of SEQ ID NOS:2-6, 9-13, and 15-35.

8. The ligand of claim 1, wherein the ligand is specific for an equine troponin I protein, a porcine troponin I protein, a bovine troponin I protein, or a combination thereof.

9. An antigen for the production of an antibody specific for a mammalian troponin molecule, wherein the antigen comprises an isolated peptide having an amino acid sequence selected from the group consisting of SEQ ID NOS:2-6, 9-13, and 15-35, wherein the antibody is not specific for an avian troponin molecule

10. An assay for detecting a mammalian troponin molecule in a sample, the assay comprising:
a) reacting the sample with a ligand that is specific for the mammalian troponin molecule and not specific for an avian troponin molecule for a time and under conditions sufficient to form a complex between the ligand and the troponin molecule; and
b) detecting the complex either directly or indirectly as a measure of the presence or amount of the troponin molecule in the sample.

11. The assay of claim 10, wherein the mammalian troponin molecule is a troponin I molecule.

12. The assay of claim 10, wherein the mammalian troponin molecule is a troponin I molecule selected from the group consisting of a slow twitch skeletal muscle troponin I molecule and a fast twitch skeletal muscle troponin I molecule.

13. The assay of claim 10, wherein the ligand is an antibody and the troponin molecule is a polypeptide.

14. The assay of claim 10, wherein the ligand is an antibody produced by immunizing an animal with a peptide having an amino acid sequence selected from the group consisting of SEQ ID NOS:2-6, 9-13, and 15-35.

15. The assay of claim 10, wherein the ligand binds to a peptide having an amino acid sequence selected from the group consisting of SEQ ID NOS:2-6, 9-13, and 15-35.

16. The assay of claim 10, wherein the ligand binds to a nucleic acid molecule encoding a peptide having an amino acid sequence selected from the group consisting of SEQ ID NOS:2-6, 9-13, and 15-35.

17. The assay of claim 10, wherein the ligand is specific for an equine troponin I protein, a porcine troponin I protein, a bovine troponin I protein or a combination thereof.

18. The assay of claim 10, wherein the sample is animal feed.

19. A method of making an antibody that is specific for a mammalian troponin molecule and not specific for an avian troponin molecule, comprising administering to an animal an immunogenic amount of a peptide having an amino acid sequence selected from the group consisting of SEQ ID NOS:2-6, 9-13, and 15-35.


SEQUENCE LISTING (35 sequences)

SEQ ID NO: 1 - protein, 178 aa, Homo sapiens
PHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 2 - protein, 182 aa, Oryctolagus sp.
MGDEEKRNRAITARRQHLKSVMLQIAATELEKEEGRREAEKQNYLAEHCPPLSLPGSMAEVQELCKQLHAKIDAAEEEKYDMEIKVQKSSKELEDMNQKLFDLRGKFKRPPLRRVRMSADAMLKALLGSKHKVCMDLRANLKQVKKEDTEKERDLRDVGDWRKNIEEKSGMEGRKKMFESES

SEQ ID NO: 3 - protein, 178 aa, Rattus sp.
MGDEEKRNRAITARRQHLKSVMLQIAATELEKEESRRESEKQNYLSEHCPPLHIPGSMSEVQELCKQLHAKIDAAEEEKYDMEVKVQKSSKELEDMNQKLFDLRGKFKRPPLRRVRMSADAMLKALLGSKHKVCMDLRANLKQVKKEDTEKERDLRDVGDWRKNIEEKSGMEGRKKMF

SEQ ID NO: 4 - protein, 182 aa, Mus sp.
MGDEEKRNRAITARRQHLKSVMLQIAATELEKEESRRESEKENYLSEHCPPLHIPGSMSEVQELCKQLHAKIDVAEEEKYDMEVKVQKSSKELEDMNQKLFDLRGKFKRPPLRRVRMSADAMLKALLGSKHKVCMDLRANLKQVKKEDTEKERDLRDVGDWRKNIEEKSGMEGRKKMFESES

SEQ ID NO: 5 - protein, 180 aa, Homo sapiens
MGDEEKRNRAITARRQHLKSVMLQIAATELEKEESRREAEKQNYLAEHCPPLHIPGSMSEVQELCKQLHAKIDAAEEEKYDMEVRVQKTSKELEDMNQKLFDLRGKFKRPPLRRVRMSADAMLKALLGSKHKVCMDLRANLKQVKKEDTEKERDLRDVGDWRKNIEEKSGMEGRKKMFES

SEQ ID NO: 6 - protein, 100 aa, Capra sp.
MGDEEKRNRAITARRQHLKSIMLQIAATELEKEEGRREAEKQNYLSGHCPPLHLPGSMSEVQELCGQLHAKIDAAEEEKYDMEVRVQKSAKELEDMNQKL

SEQ ID NO: 7 - protein, 183 aa, Gallus sp.
MSDEEKKRRAATARRQHLKSAMLQLAVTEIEKEAAAKEVEKQNYLAEHCPPLSLPGSMQELQELCKKLHAKIDSVDEERYDTEVKLQKTNKELEDLSQKLFDLRGKFKRPPLRRVRMSADAMLRALLGSKHKVNMDLRANLKQVKKEDTEKEKDLRDVGDWRKNIEEKSGMEGRKKMFEAGES

SEQ ID NO: 8 - protein, 183 aa, Coturnix sp.; identical to SEQ ID NO: 7

SEQ ID NO: 9 - protein, 187 aa, Mus sp.
MPEVERKSKITASRKLMLKSLMLAKAKECWEQEHEEREAEKVRYLSERIPTLQTRGLSLSALQDLCRELHAKVEVVDEERYDIEAKCLHNTREIKDLKLKVLDLRGKFKRPPLRRVRVSADAMLRALLGSKHKVSMDLRANLKSVKKEDTEKERPVEVGDWRKNVEAMSGMEGRKKMFDAAKSPTSQ

SEQ ID NO: 10 - protein, 187 aa, Rattus sp.
MPEVERKSKITASRKLMLKSLMLAKAKECWEQEHEEREAEKVRYLSERIPTLQTRGLSLSALQDLCRELHAKVEVVDEERYDIEAKCLHNTREIKDLKLKVLDLRGKFKRPPLRRVRVSADAMLRALLGSKHKVSMDLRANLKSVKKEDTEKERPVEVGDWRKNVEAMSGMEGRKKMFDAAKSPTLQ

SEQ ID NO: 11 - protein, 187 aa, Homo sapiens
MPEVERKPKITASRKLLLKSLMLAKAKECWEQEHEEREAEKVRYLAERIPTLQTRGLSLSALQDLCRELHAKVEVVDEERYDIEAKCLHNTREIKDLKLKVMDLRGKFKRPPLRRVRVSADAMLRALLGSRHKVSMDLRANLKSVKKEDTEKERPVEVGDWRKNVEAMSGMEGRKKMFDAAKSPTSQ

SEQ ID NO: 12 - protein, 184 aa, Oryctolagus sp.
PEVERKSKITASRKLLKSLMLAKAKECQQEHEAREAEKVRYLAERIPALQTRGLSLSALQDLCRQLHAKVEVVDEERYDIEAKCLHNTREIKDLKLKVLDLRGKFKRPPLRRVRVSADAMLRALLGSKHKVSMDLRANLKSVKKEDTEKERPVEVGDWRKNVEAMSGMEGRKKMFDAAKSPTSQ

SEQ ID NO: 13 - protein, 108 aa, Capra sp.
MPEVERKPKITASRKLLLKSLMLARAKECWDQELEEREAEKKRYLAERVPSLQTRGLSLSALQDLCRDLHAKVEVVDEERYDIEAKCLHNTREIKDLKLKVLDLRGKF

SEQ ID NO: 14 - protein, 186 aa, Coturnix sp.
MPEVERKSKITASRKLLLKSLMLAKAKEEWEQEIVDKQSEKERYLSERITPLHTSGLSLSQLQDLCRELHEKVEVTDEERYDIEAKCNHNTREIKDLKLKVLDLRGKFKRPPLRRVRVSADAMLRALLGSKHKVSMDLRANLKSVKKEDTEKERPVEVGDWRKNVEAMSGMEGRKKMFDAAKSPTG

SEQ ID NO: 15 - protein, 39 aa, Mus sp.
VMLQIAATELEKEESRRESEKENYLSEHCPPLHIPGSMS

SEQ ID NO: 16 - protein, 39 aa, Capra sp.
IMLQIAATELEKEEGRREAEKQNYLSGHCPPLHLPGSMS

SEQ ID NO: 17 - protein, 20 aa, Rattus sp.
VMLQIAATELEKEESRRESE

SEQ ID NO: 18 - protein, 20 aa, Capra sp.
IMLQIAATELEKEEGRREAE

SEQ ID NO: 19 - protein, 14 aa, Unknown
  Organism is Rattus sp. and Mus sp.
SEHCPPLHIPGSMS

SEQ ID NO: 20 - protein, 14 aa, Artificial Sequence
  Synthetic
SGHCPPLHIPGSMS

SEQ ID NO: 21 - protein, 10 aa, Unknown
  Organism is Rattus sp. and Mus sp.
EKEESRRESE

SEQ ID NO: 22 - protein, 10 aa, Capra sp.
EKEEGRREAE

SEQ ID NO: 23 - protein, 37 aa, Rattus sp.
VQELCKQLHAKIDAAEEEKYDMEVKVQKSSKELEDMN

SEQ ID NO: 24 - protein, 37 aa, Capra sp.
VQELCGQLHAKIDAAEEEKYDMEVRVQKSAKELEDMN

SEQ ID NO: 25 - protein, 22 aa, Unknown
  Organism is Homo sapiens, Rattus sp. and Mus. sp.
VQELCKQLHAKIDAAEEEKYDM

SEQ ID NO: 26 - protein, 22 aa, Capra sp.
VQELCGQLHAKIDAAEEEKYDM

SEQ ID NO: 27 - protein, 12 aa, Unknown
  Organism is Oryctolagus sp., Rattus sp. and Homo sapiens
KQLHAKIDAAEE

SEQ ID NO: 28 - protein, 12 aa, Capra sp.
GQLHAKIDAAEE

SEQ ID NO: 29 - protein, 49 aa, Homo sapiens
ECWEQEHEEREAEKVRYLAERIPTLQTRGLSLSALQDLCRELHAKVEVV

SEQ ID NO: 30 - protein, 49 aa, Artificial Sequence
  Synthetic
ECWEQELEEREAEKKRYLAERIPTLQTRGLSLSALQDLCRDLHAKVEVV

SEQ ID NO: 31 - protein, 7 aa, Unknown
  Organism is Rattus sp., Mus sp., Homo sapiens, Oryctolagus sp., and Capra sp.
HAKVEVV

SEQ ID NO: 32 - protein, 17 aa, Unknown
  Organism is Mus sp., Rattus sp., and Homo sapiens
ECWEQEHEEREAEKVRY

SEQ ID NO: 33 - protein, 17 aa, Artificial Sequence
  Synthetic
ECWEQELEEREAEKKRY

SEQ ID NO: 34 - protein, 7 aa, Unknown
  Organism is Rattus sp., Mus sp., and Homo sapiens
IPTLQTR

SEQ ID NO: 35 - protein, 7 aa, Artificial Sequence
  Synthetic
IPSLQTR